SEQUENCE LISTING (1 sequence)

SEQ ID NO: 1 - protein, 34 aa, synthetic construct
DPFFKVPVNKLAAAVSNFGYDLYRVRSSTSPTTN

(19) United States
(12) Patent Application Publication
(10) Pub. No.: US 2026/0241015 A1
(43) Pub. Date: Aug. 20, 2026

(54) EFFICIENT DRUG DELIVERY PLATFORM FOR TREATING EYE DISEASES

(71) Applicant: NEXTHERA CO.,LTD., Busan (KR)

(72) Inventors: Kug-Hwan ROH, Busan (KR); Nayoung LIM, Busan (KR); Yousong LEE, Busan (KR); Min Chang CHOI, Busan (KR)

(73) Assignee: NEXTHERA CO.,LTD., Busan (KR)

(21) Appl. No.: 19/165,588

(22) PCT Filed: Dec. 21, 2023
(86) PCT No.: PCT/KR2023/021262

§ 371 Date: Sep. 16, 2025

(30) Foreign Application Priority Data
Dec. 27, 2022 (KR) 10-2022-0186319
Dec. 14, 2023 (KR) 10-2023-0182060

(51) Int. Cl. A61K 47/64; A61K 9/00; A61K 38/39; A61P 27/04

(52) U.S. Cl. CPC A61K 47/64; A61K 9/0048; A61K 38/39; A61P 27/04

(57) ABSTRACT

The present invention relates to an efficient drug delivery platform for treating eye diseases, wherein it has been identified that when a compound is conjugated to PEDF34, is mixed with type I collagen and applied to the eyes, the compound conjugated to PEDF34 is efficiently delivered into the eyes, and substance delivery is much better than when the compound is used alone or the compound conjugated to PEDF34 is used without type I collagen.

### TECHNICAL FIELD

The present disclosure relates to a drug delivery platform utilizing collagen in order to efficiently deliver an effective substance for treating an ocular disease.

### BACKGROUND ART

Recently, the increase in geriatric diseases due to the aging in Korea has emerged as a social issue, and age-related macular degeneration is particularly a common and serious disease that is called the ‘eye plague’ as it occurs in one out of three people in their 70s. Macular degeneration is a condition in which new blood vessels grow abnormally and damage the macula to cause effects on vision, and in the case of wet macular degeneration, which may lead to blindness, it is estimated that there are about 10,000 patients in Korea alone. Treatment methods for macular degeneration conducted currently include laser photocoagulation for a case where boundaries of the area where degeneration has occurred are clearly identified, and other treatment methods, such as photodynamic therapy, antibody injections, and vitreous removal (vitrectomy), have been proposed, but there is still no complete cure.

Intraocular drug delivery is largely carried out through invasive and non-invasive methods, and a representative invasive method of drug delivery is intravitreal drug injection. When injecting drugs by intravitreal injection, frequent injections are required to maintain the short half-life of the delivered drug due to the active elimination mechanism occurring within the vitreous cavity, which may cause side effects such as retinal detachment, retinal hemorrhage, and endophthalmitis. In addition, eye drops are the most representative non-invasive method, and the use thereof ensures excellent convenience and safety. However, the conventional method of administering eye drops provides a poor efficacy compared to the amount used due to a short retention time of the drug on the corneal surface, and the drug washed off from the corneal surface circulates throughout the body with tears to lead to side effects.

A pigment epithelium-derived factor (PEDF), a protein exhibiting anti-angiogenic effects, is a substance considered as a treatment for various diseases caused by angiogenesis, and currently, research is being conducted to utilize PEDF as a treatment for macular degeneration, bearing a problem that it requires repeated injections due to its short half-life with a narrow range of concentration applied for disease treatment.

Accordingly, in order to resolve the problems in treating ocular diseases as described above, there is a need to develop a more effective drug delivery platform for treating ocular diseases.

### DISCLOSURE OF THE INVENTION

### Technical Goals

An object of the present disclosure is to determine whether a therapeutic drug for an ocular disease is delivered into the eye by conjugating the therapeutic drug for an ocular disease to a pigment epithelium-derived factor (PEDF) peptide and mixing it with a collagen type I solution, to provide a stable intraocular drug delivery platform.

### Technical Solutions

To achieve the above object, the present disclosure provides a composition for intraocular drug delivery, including a pigment epithelium-derived factor (PEDF) peptide, to which a therapeutic drug for an ocular disease is conjugated, and collagen as active ingredients.

### Advantageous Effects

The present disclosure relates to an efficient drug delivery platform for treating an ocular disease, and more particularly, it was found that when a compound is conjugated to PEDF34 and mixed with collagen type I to be applied to the eye, the compound conjugated to PEDF34 is efficiently delivered into the eye, showing remarkably superior substance delivery compared to cases where the compound is used alone or when the compound conjugated to PEDF34 is used without collagen type I. Therefore, this method may be utilized as an efficient drug delivery platform for an intraocular disease.

### BRIEF DESCRIPTION OF DRAWINGS

FIG. 1 shows results of identifying the transfer of a compound into an intraocular tissue through analysis with confocal microscopy by preparing frozen sections of mouse eyes of a group that received eye drops of a composition (NT-101-AF) prepared by mixing AF647 conjugated to a 34-mer pigment epithelium-derived factor peptide (PEDF34) with collagen type I and a group that received AF647 eye drops only.

FIG. 2 shows results of comparing a group that received eye drops of a composition (NT-101-AF) prepared by mixing AF647 conjugated to a 34-mer pigment epithelium-derived factor (PEDF34-AF) peptide with collagen type I and a group that received eye drops of PEDF34-AF (a 34-mer pigment epithelium-derived factor peptide conjugated with AF647), showing the transfer of the compound into an intraocular tissue through analysis with a confocal microscope by preparing frozen sections of the mouse eyes of each group.

FIG. 3 shows outcome images taken by a customized intravital microscope (IVM-FC) to visualize drug delivery to the retina by applying eye drops of a composition (NT-101-FITC) prepared by mixing FITC conjugated to a 34-mer pigment epithelium-derived factor (PEDF34) peptide with collagen type I, presenting photographs at each time point before and after eye drop instillation as well as quantified results of an area of drug delivery to the retina.

### BEST MODE FOR CARRYING OUT THE INVENTION

The present disclosure was completed by identifying, using two substances having different structures, Alexa Fluor 647 (AF647) and fluorescein isothiocyanate (FITC) as examples of compounds, that the substances were able to be efficiently and stably delivered into the eye when these substances are conjugated to 34-mer pigment epithelium-derived factor (PEDF34) peptide and then mixed with collagen type I to be used as a mixture (NT-101), thereby providing a potential of being usefully utilized as an efficient drug delivery platform for treating an ocular disease.

The present disclosure provides a composition for intraocular drug delivery, including a pigment epithelium-derived factor (PEDF) peptide, to which a therapeutic drug for an ocular disease is conjugated, and collagen as active ingredients.

Preferably, the PEDF peptide may consist of an amino acid set forth in SEQ ID NO: 1 (DPFFK VPVNK LAAAV SNFGY DLYRV RSSTS PTTN), but is not limited thereto. The 34-mer PEDF peptide of the SEQ ID NO: 1 is presented as “PEDF34” in the present specification.

Preferably, the collagen may be a 0.01 to 0.5% collagen type I solution, but is not limited thereto.

Preferably, the therapeutic drug for an ocular disease may be any one selected from the group consisting of cyclosporine, lifitegrast, diquafosol, fluorometholone, loteprednol, prednisolone, tacrolimus, moxifloxacin, levofloxacin, cephalosporin, vancomycin, acyclovir, ganciclovir, natamycin, voriconazole, timolol, betaxolol, brimonidine, brinzolamide, latanoprost, travoprost, tafluprost, bimatoprost, pilocarpine, dorzolamide, anti-vascular endothelial growth factor (anti-VEGF), and triamcinolone, but is not limited thereto.

Preferably, the composition may target, but is not limited to, the retina within the eye.

Preferably, the composition may be in the form of eye drops, but is not limited thereto.

As used herein, “NT-101-AF” refers to an eye drop formulation prepared by mixing AF647-conjugated epithelium-derived factor with collagen type I, and “NT-101-FITC” refers to an eye drop formulation prepared by mixing FITC-conjugated epithelium-derived factor peptide with collagen type I.

In the present disclosure, the composition for drug delivery may further include one or more pharmaceutically acceptable carriers, excipients, or diluents in addition to the active ingredient of the present disclosure.

Additionally, the term “pharmaceutically acceptable” as used herein refers to a state that is physiologically acceptable without typically causing an allergic reaction such as dizziness or a similar reaction when administered to humans.

Examples of the carriers, excipients, and diluents may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

In addition, the composition for drug delivery may further include fillers, anticoagulants, lubricants, wetting agents, fragrances, emulsifiers, and preservatives.

### Modes for Carrying Out the Invention

Hereinafter, the present disclosure will be described in more detail through examples. These examples are only intended to illustrate the present disclosure more specifically, and it will be apparent to those skilled in the art that the scope of the present disclosure is not limited by these examples in accordance with the gist of the present disclosure.

### <Preparation Example> Preparation of Eye Drop Compositions

With regard to a composition of the present disclosure, an eye drop was prepared in a type of NT-101 by conjugating a compound (AF647, FITC) to a pigment epithelium-derived factor (PEDF) peptide 34-mer (PEDF34) and mixing it with collagen type I.

1. An Eye Drop Composition with AF647 Conjugated to a Pigment Epithelium-Derived Factor Peptide

The conjugation of PEDF34 and AF647 was performed with reference to the conjugation method provided by ABCAM.

1 mL of 1 mg/mL (265.73 μM) PEDF34 solution was prepared, and then 100 μL of modifier reagent was added and mixed. Afterwards, the mixture solution was added to the freeze-dried AF647 powder, gently suspended, and then kept in a dark room for 15 minutes, followed by conjugation. After 15 minutes, 100 μL of Quencher reagent was added and mixed, and the mixture was kept at room temperature for 5 minutes before being used to prepare eye drops.

2. An Eye Drop Composition with FITC Conjugated to a Pigment Epithelium-Derived Factor Peptide

Anygen Co., LTD. was commissioned to synthesize a sample conjugated with pigment epithelium-derived factor peptide (PEDF34) and FITC, and the synthesized PEDF34-FITC was prepared at a concentration of 10 mM (42.65 mg/mL) and subsequently used in the manufacture of eye drops.

3. Preparation of Eye Drops by Mixing AF647-Conjugated Pigment Epithelium-Derived Factor Peptide with Collagen Type I

To confirm the intraocular transport of AF647-conjugated epithelium-derived factor peptide (PEDF34-AF), 50 μM and 133 μM eye drops (NT-101-AF) were prepared by mixing PEDF34-AF with 0.1% collagen type I and administered to mouse eyes.

The preparation of 50 μM eye drops was carried out by diluting PEDF34-AF prepared at a concentration of 1 mg/mL (265.73 μM) to 100 μM and mixing it with 0.2% collagen type I solution in a 1:1 ratio to prepare the 50 μM eye drop (NT-101-AF) including PEDF34-AF mixed with 0.1% collagen type I.

The preparation of 133 μM eye drops was carried out by mixing PEDF34-AF prepared at a concentration of 1 mg/mL (265.73 μM) with 0.2% collagen type I solution in a 1:1 ratio to prepare the 133 μM eye drop (NT-101-AF) including PEDF34-AF mixed with 0.1% collagen type I.

4. Preparation of Eye Drops by Mixing FITC-Conjugated Pigment Epithelium-Derived Factor Peptide with Collagen Type I

To photograph the delivery phenomenon of FITC-conjugated epithelium-derived factor peptide (PEDF34-FITC) to the intraocular retinal tissue, a 133 μM eye drop (NT-101-FITC) was prepared by mixing PEDF34-FITC with 0.1% collagen type I and administered to mouse eyes, followed by photographing.

The preparation of 133 μM eye drops was carried out by diluting PEDF34-FITC prepared at a concentration of 42.65 mg/mL (10 mM) to 266 μM and mixing it with 0.2% collagen type I solution in a 1:1 ratio to prepare the 133 μM eye drop (NT-101-FITC) including PEDF34-FITC mixed with 0.1% collagen type I.

### <Example 1> Identification of Intraocular Delivery of AF647 Conjugated to a Pigment Epithelium-Derived Factor Peptide

The eye drops of the present disclosure prepared according to the above Preparation Example were instilled into the eyes of mice to detect whether they moved within the eyes. The animal model used in the experiment was a 5-week-old C57BL/6 mouse, purchased from Nara Biotech, and used in this experiment when it was 6 to 8 weeks old after going through a breeding adaptation period.

Mice were anesthetized by intraperitoneal injection of 100 mg/kg ketamine (Huons, SEONGNAM-SI, GYEONGGI-DO, Korea) and 10 mg/kg xylazine (BAYER, Leverkusen, Germany), and after anesthesia, 10 μL of each of the 133 μM eye drop (NT-101-AF) prepared by mixing AF647-conjugated epithelium-derived factor (PEDF34-AF) peptide with 0.1% collagen type I prepared in Preparation Example 3 and AF647 were instilled into the mouse eyes. After 30 minutes, the mice were euthanized to remove the eyes and then fixed in 10% formalin for 10 minutes to prepare OCT blocks. Afterwards, frozen sections (10 μm) were prepared and stained with DAPI, and the distribution of AF647 in ocular tissue was detected using a confocal microscope.

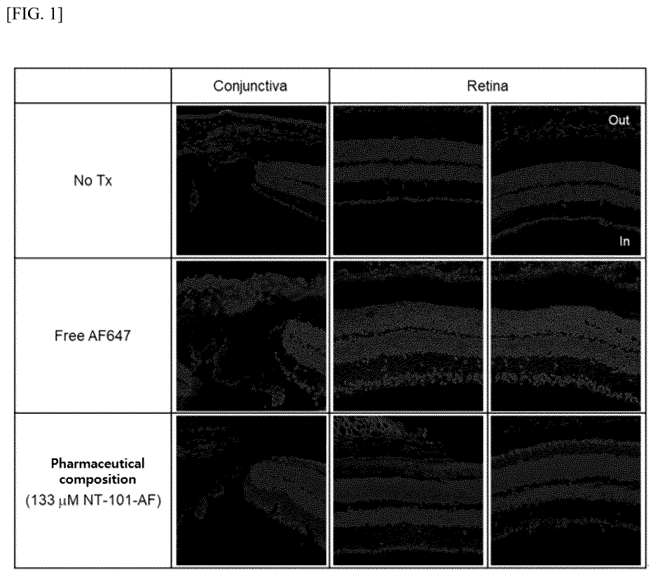

As shown in FIG. 1, the 133 μM eye drops (NT-101-AF) including AF647-conjugated epithelium-derived factor (PEDF34-AF) peptide mixed with 0.1% collagen type I prepared in the Preparation Example 3 showed increased intraocular delivery compared to a group injected with the eye drop of AF647 only, demonstrating that the mixture of PEDF34 and collagen type I may be utilized as a retinal delivery platform.

### <Example 2> Verification of an Increase in Intraocular Delivery of AF647 Conjugated to a Pigment Epithelium-Derived Factor Peptide Composition Using a Retinal Delivery Platform

From the results of Example 1 above, it was found that intraocular delivery was possible using the eye drops (NT-101-AF) including AF647-conjugated epithelium-derived factor (PEDF34-AF) peptide mixed with 0.1% collagen type I, i.e., a retinal delivery platform, compared to the group that received the AF647 eye drop only, and it was attempted to identify whether intraocular delivery increased when the retinal delivery platform was applied.

Mice were anesthetized by intraperitoneal injection of 100 mg/kg ketamine (Huons, SEONGNAM-SI, GYEONGGI-DO, Korea) and 10 mg/kg xylazine (BAYER, Leverkusen, Germany), and after anesthesia, 10 μL of each of the 50 μM eye drop (NT-101-AF) including AF647-conjugated epithelium-derived factor peptide mixed with 0.1% collagen type I prepared in Preparation Example 3 and 50 μM AF647-conjugated epithelium-derived factor (PEDF34-AF) peptide alone were instilled into the mouse eyes. 15, 30, 45, and 60 minutes after eye instillation, each mouse was euthanized to remove the eyes and then fixed in 10% formalin for 10 minutes to prepare OCT blocks. Afterwards, frozen sections (10 μm) were prepared and stained with DAPI, and the distribution of AF647 in ocular tissue was detected using a confocal microscope.

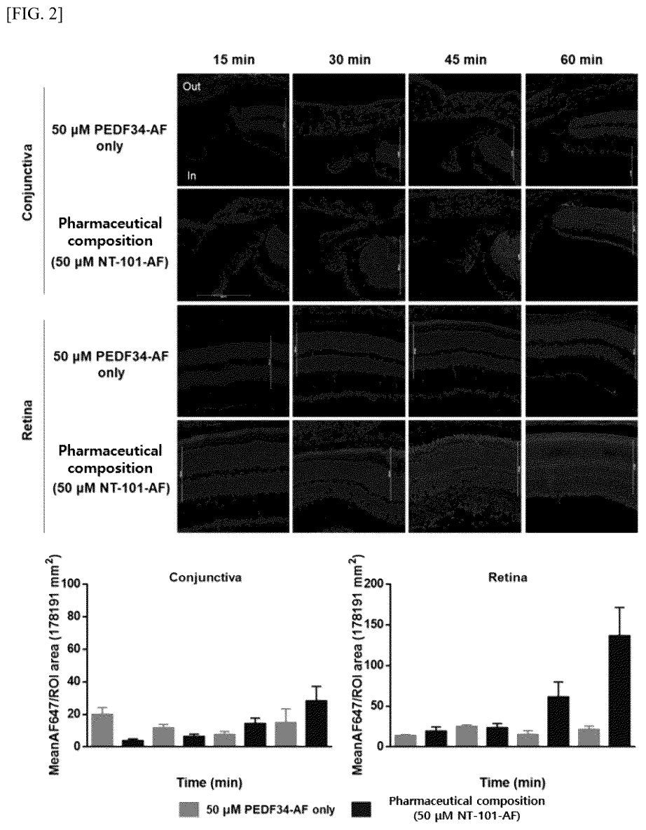

As shown in FIG. 2, in the conjunctiva, the group (PEDF34-AF) administered with 50 μM AF647-conjugated epithelium-derived factor peptide alone showed the highest distribution at 15 minutes after instillation, but showed a tendency to decrease over time, while the group (NT-101-AF) administered with the 50 μM eye drop prepared by mixing AF647-conjugated epithelium-derived factor peptide with 0.1% collagen type I showed an increase in the tissue distribution of AF647 over time. In addition, in the retina, the group (PEDF34-AF) administered with 50 μM AF647-conjugated epithelium-derived factor peptide alone showed no significant change over time, but the group (NT-101-AF) administered with the 50 μM eye drop prepared by mixing AF647-conjugated epithelium-derived factor peptide with 0.1% collagen type I showed a rapid increase in the tissue distribution of AF647 over time.

### <Example 3> In-Vivo Imaging of the Intraocular Delivery Pattern of FITC Conjugated to a Pigment Epithelium-Derived Factor Peptide Composition

In this Example, it was attempted to identify intraocular delivery and delivery patterns using a retinal delivery platform by utilizing FITC, a compound with a different structure from the AF647 compound identified in Examples 1 and 2.

The eye drop (NT-101-FITC) including the FITC-conjugated pigment epithelium-derived factor (PEDF34-FITC) mixed with collagen type I prepared in Preparation Example 4 was instilled into the mouse eye followed by ocular imaging at 10, 20, and 30 minutes, and then imaging was performed immediately before instillation of the eye drop for comparison. The anti-CD31 antibody was labeled red with a separate label and injected intravenously with tetramethylrhodamine-dextran (TMR-Dex). Imaging was performed at the same location at each time point to observe whether the delivery takes place in the retina, and IVM-FC equipment was used for retina imaging (IVIM Tech). The quantitative analysis method was carried out by photographing 5 to 6 areas per subject, and the relative value of FITC fluorescence signal intensity in the retinal tissue was presented compared to the time point immediately before eye instillation.

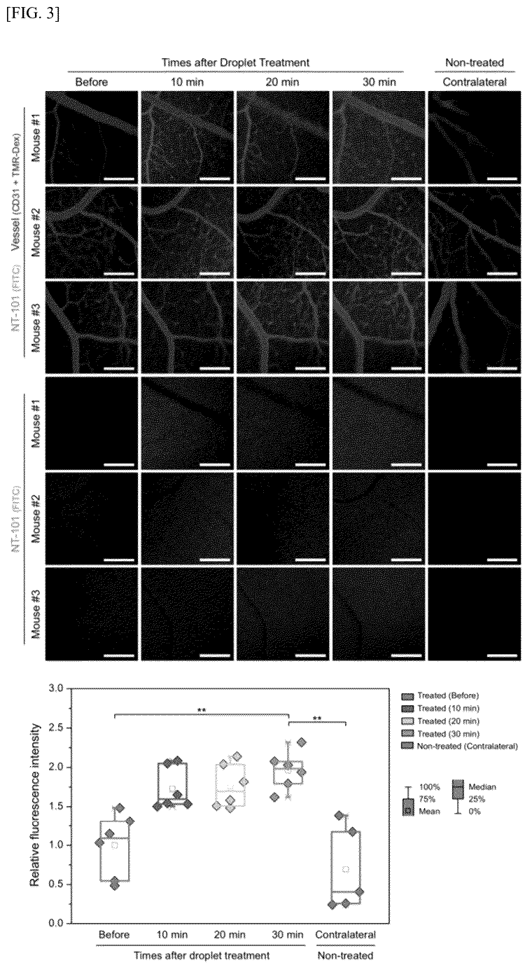

As shown in FIG. 3, when the eye drop (NT-101-FITC) prepared by mixing the FITC-conjugated pigment epithelium-derived factor with collagen type I prepared according to Preparation Example 4 was instilled to the eye, the FITC observed within the retinal tissue gradually increased over time.

In summary of the above results and the results from Examples 1, 2, and 3, it was determined that the intraocular transfer of a compound conjugated to an epithelium-derived factor peptide may be increased when a retinal delivery platform is utilized to deliver the compound through instillation of the eye drops (NT-101 method) prepared by mixing the pigment epithelium-derived factor peptide with collagen type I.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

1. A method for enhancing an intraocular drug delivery for an ocular disease to a subject, comprising:
administering a composition comprising a pigment epithelium-derived factor (PEDF) peptide, to which a therapeutic drug for the ocular disease is conjugated, and collagen as active ingredients to a subject.

2. The method of claim 1, wherein the PEDF peptide comprises an amino acid set forth in SEQ ID NO: 1.

3. The method of claim 1, wherein the collagen is a 0.01 to 0.5% collagen type I solution.

4. The method of claim 1, wherein the therapeutic drug for an ocular disease is any one selected from the group consisting of cyclosporine, lifitegrast, diquafosol, fluorometholone, loteprednol, prednisolone, tacrolimus, moxifloxacin, levofloxacin, cephalosporin, vancomycin, acyclovir, ganciclovir, natamycin, voriconazole, timolol, betaxolol, brimonidine, brinzolamide, latanoprost, travoprost, tafluprost, bimatoprost, pilocarpine, dorzolamide, anti-vascular endothelial growth factor (anti-VEGF), and triamcinolone.

5. The method of claim 1, wherein the composition targets the retina in the eye.

6. The method of claim 1, wherein the composition is in the form of an eye drop.
